(19) United States
(12) Patent Application Publication
(10) Pub. No.: US 2007/0077639 A1
(43) Pub. Date: Apr. 5, 2007

(54) Estimation of activity or inhibition of processes involved in nucleic acid modification using chemiluminescence quenching

(76) Inventors: Ian Weeks, Cardiff (GB); Richard C. Brown, Cardiff (GB); Andrew Morby, Cardiff (GB); Colin Berry, Cardiff (GB)

Correspondence Address: GEN PROBE INCORPORATED, 10210 GENETIC CENTER DRIVE, Mail Stop #1 / Patent Dept., SAN DIEGO, CA, 92121, US

(21) Appl. No.: 10/570,176

(22) PCT Filed: Aug. 26, 2004
(86) PCT No.: PCT/GB04/03633

§ 371 Date: Dec. 14, 2006

(30) Foreign Application Priority Data
Aug. 29, 2003 (GB) 0320235.5

(51) Int. Cl. C12N 9/94

(52) U.S. Cl. 435/186

(57) ABSTRACT

A method for determining the activity of a substance capable of altering the structure of a nucleic acid molecule from a first to a second state which is based upon the use of labelled nucleic acid molecules and/or complementary oligonucleotides. The label is a chemiluminescent molecule and a corresponding quencher molecule whose optical properties are different depending upon whether the nucleic acid molecule exist in said first or second state.

### FIELD OF THE INVENTION

This invention relates to means of estimating the activity or inhibition of activity of processes involved in modification of genetic material, said means being based on the use of labelled nucleic acid molecules or oligonucleotides wherein the labels used are chemiluminescent molecules whose optical properties are different depending upon whether the labelled nucleic acid molecules or oligonucleotides are present in the form of single stranded or multiple stranded nucleic acid; and, further the use of said means for drug discovery.

### BACKGROUND OF THE INVENTION

The replication, recombination, repair and other modifications of deoxyribonucleic acid (DNA) and ribonucleic acid (RNA) molecules all involve changes in the structure of genetic material and are of fundamental importance to all living organisms. Examples of such modifications are enzymatic reactions where the enzymes are ligases, nucleases, integrases, transposases, helicases, polymerases, topoisomerases, primases, reverse transcriptases and gyrases. Such enzymes are ubiquitous and required for most aspects of nucleic acid metabolism. The importance of assessing the activity of these enzymes has therefore led to attempts to develop assay systems for such purposes. Of particular, but not exclusive, importance is the ability to monitor bacterial or viral enzyme activity in the screening of novel compounds for their ability to inhibit these enzymes and hence demonstrate anti-bacterial or anti-viral properties. Also of importance are non-protein molecules which similarly act upon nucleic acid to alter its structural characteristics, such molecules being exemplified by enediynes, ribozymes and aptamers.

### DESCRIPTION OF THE PRIOR ART

Current assays for assessing the activity of these enzymes (e.g. ligases, FIG. 1) involve measuring the production of enzyme intermediates or the structural characteristics of the substrate and/or product. The majority of these assays employ radioactivity, necessitating additional experimental precautions and incurring costs for waste disposal. Recent assays have attempted to replace the use of radioactivity with fluorescent labels for DNA substrates but they lack the necessary sensitivity of detection to enable them to be widely used. Alternatively, biological assays for DNA ligase activity have been developed but they are time consuming (at least 2 days), laborious and qualitative rather than quantitative. A more rapid biological assay has been described (U.S. Pat. No. 5,976,806) but this involves the use of coupled transcription-translation systems with expression of a reporter gene product (e.g. luciferase), in addition to DNA ligase, making this assay unsuitable for the high-throughput screening of potential pharmaceutical compounds.

Assays for helicase (FIG. 2) have been described which exploit the unwinding of double stranded nucleic acid. In one example (U.S. Pat. No. 5,958,696), a solid-phase derivative of a double-stranded nucleic acid is prepared in which one of the strands is labelled with a radioisotope. Helicase activity is detected by its ability to release the labelled strand into the soluble phase which can be separated and measured. In a further example, use is made of the ability of certain markers to preferentially associate with double stranded nucleic acid as opposed to single stranded nucleic acid. Thus the marker will not associate with material which is unwound by helicase activity.

Further, the use of direct chemiluminescent-labelled oligonucleotide probes has been described for the quantification of RNA in infectious organisms (U.S. Pat. No. 5,283,174, U.S. Pat. No. 5,399,491) and the use of chemiluminescent donor/acceptor pairs has been described for use in the labelling of oligonucleotide probes for the detection and/or quantitation of nucleic acid targets (WO 01/42497 A2).

The substances of interest in the present invention all result in a structural change of a nucleic acid substrate to yield a nucleic acid product and therefore there is a need to find a probe that can discriminate between the nucleic acids that constitute the substrate and product molecules associated with the substances of interest mentioned herein.

The aim of the present invention therefore is to provide means of measuring the activity of substances, such as enzymes or non-protein molecules, involved in nucleic acid metabolism and particularly in the repair and replication of genetic material which means employ the use of chemiluminescence emitter/quencher labelled nucleic acid sequences capable of differentiating the substrate and product molecules appropriate to these substances; and, further the use of said means in drug discovery.

Reference herein to the term ‘enzyme activity’ or ‘substance activity’ includes reference to an increase, decrease or no change in activity.

Reference herein to the term ‘substrate’ includes references to a molecule that the substance of interests acts upon.

Reference herein to the term ‘product’ includes reference to a molecule that is produced following the activity of the substance of interest.

### SUMMARY OF THE INVENTION

We have developed assays to measure the activity of enzymes or other substances involved in nucleic acid metabolism which are simple, rapid and robust. Whilst useful in many situations where the assessment of such activity is required, these properties make such assays particularly suitable for the screening of putative anti-bacterial and anti-viral compounds capable of inhibiting the enzyme activity. It will also be appreciated that the ability to determine the relative amounts of substrate and product also has utility in situations where the structural change, normally brought about enzymatically, is brought about non-enzymatically. In this way the principles taught herein may be applied to any situation in which it is desired to determine the relative amounts of modified and unmodified nucleic acid. For example, such a situation would include an instance where ultraviolet rays or radio waves are used to change the structure of a nucleic acid molecule.

According to a first aspect of the invention there is therefore provided a method for determining the activity of a substance capable of altering the structure of a nucleic acid from a first state to a second state comprising the steps of:

(a) providing in a test sample:
(i) said substance;
(ii) said nucleic acid; and, optionally,
(iii) one or more oligonucleotides complementary, at least in part, to said nucleic acid when in said first or second state;
wherein said nucleic acid and/or said oligonucleotide is labelled with at least one chemiluminescent molecule and/or at least one quencher molecule capable of attenuating chemiluminescence from said chemiluminescent molecule, said chemiluminescent and quencher molecules being arranged so that the interaction therebetween changes according to whether said nucleic acid is in said first or second state whereby in one of said first or second states said chemiluminescence is substantially attenuated;

(b) monitoring the chemiluminescence emission of said chemiluminescent molecule; and, optionally,

(c) comparing said emission with that corresponding to the absence of said substance.

It may therefore be apparent to one skilled in the art that the invention involves, what might be termed, a bimolecular system wherein a first molecule carries said chemiluminescent molecule and a second molecule carries said quencher molecule. For example, in one example, one of said molecules may be said oligonucleotide and a second of said molecules may be said nucleic acid. Alternatively, said bimolecular system may be interpreted to mean two strands of nucleic acid wherein one of said strands is provided with said chemiluminescent molecule and a second of said strands is provided with said quencher molecule.

Thus, the method of the invention involves the use of at least one nucleic acid or oligonucleotide sequence labelled with a chemiluminescent molecule in such a way that the chemiluminescent molecule comes into close proximity with a quencher molecule such that the optical properties of the chemiluminescent molecule differ depending on whether or not the nucleic acid or oligonucleotide, to which the chemiluminescent label is preferably attached, is hybridised to form a duplex with a complementary nucleic acid sequence.

In a preferred embodiment of the invention, said oligonucleotide sequence is designed to hybridise to a selected enzyme or non-protein substrate nucleic acid, or its corresponding product, so that binding therebetween can be used to monitor a reaction. Additionally, the oligonucleotide may be used to monitor the conversion of a nucleic acid from a first to a second state by other means. This is achieved by ensuring that the oligonucleotide is designed to distinguish between said first and second states. This selectivity enables the process of a reaction to be monitored as a molecule is converted from a first to a second state by the process.

Most ideally conversion of said nucleic acid from a first to a second state is undertaken enzymatically and thus the method is used to determine the activity of any one or more of the following enzymes: ligase, nuclease, integrase, transposase, helicase, polymerase, topoisomerase, primase, reverse transcriptase and gyrase.

Additionally, or alternatively, said oligonucleotide sequence is designed to hybridise to a selected non-protein substrate nucleic acid or its corresponding product.

Additionally, or alternatively still, said oligonucleotide sequence is designed to hybridise to a selected substrate or product nucleic acid that is converted from a first to a second state by physical means such as electromagnetic energy and particularly electromagnetic energy in the form of ultraviolet light or radio waves.

Enediynes are naturally occurring non-protein organic molecules (e.g. calicheamicin and esperamicin) that behave as restriction endonucleases. They have the ability to cleave duplex nucleic acid, and it is this ability to convert a nucleic acid molecule from a first to a second state that enables these molecules to be included within the scope of this invention.

Similarly, it has been shown that ultraviolet rays and radio waves have the ability to act upon nucleic acid in order to convert it from a first to a second state.

It therefore follows that the above substances (including electromagnetic energy) fall within the scope of the invention since they are able to convert a nucleic acid molecule from a first to a second state and thus, using the technology described herein, the activity of these substances can be assayed. Furthermore, using the invention described herein the presence of these substances, and thus the presence of their activity within a sample, can also be identified. Furthermore, given the ability of these substances to alter the molecular structure of a nucleic acid from a first to a second state it also follows that, using the invention described herein, it is possible to screen for molecules that regulate the activity of these substances and so identify molecules or agents which are active pharmacologically as agonists or antagonists thereof.

In the instance where the method is used to monitor the activity of more than one substance part (a) thereof involves providing in said test sample a plurality of substances, their corresponding nucleic acid(s) and, optionally, a plurality of oligonucleotides, said nucleic acid(s) and/or said oligonucleotides having attached thereto a plurality of different chemiluminescent molecules and their corresponding quencher molecules wherein the attachment of selected different chemiluminescent molecules and their quencher molecules to selected nucleic acid(s) and/or oligonucleotides is designed to monitor a particular reaction within said test sample.

It will be readily apparent to those skilled in the art that chemiluminescent molecules and their corresponding quencher molecules are selected so as to maximise the signal therefrom and also to maximise the different signals therebetween. So, for example, in one embodiment where more than one enzyme, or other nucleic acid modifying reaction, is to be monitored an oligonucleotide complementary to the substrate, or product, of a first enzyme or modifier is labelled with a first chemiluminescent molecule and its corresponding quencher and a second oligonucleotide complementary to the substrate, or product, of a second enzyme or modifier is also provided and this oligonucleotide is labelled with a chemiluminescent molecule, and corresponding quencher, which is distinct from the first whereby two chemiluminescent signals can be simultaneously monitored. Alternatively, either of said first or second chemiluminescent molecules may be provided on said nucleic acid substrate, or the enzyme or modifier product thereof, and said corresponding quencher may be provided on said oligonucleotide, or vice versa. Alternatively still, said oligonucleotide may be omitted from said test sample and said substrate nucleic acid, or the reaction product thereof, may be labelled with said chemiluminescent molecule and said quencher.

The quencher molecule may be situated on the same nucleic acid strand or on a different nucleic acid strand with respect to the chemiluminescent emitter.

In summary in the direct assay methods referred to below the label moieties may form part of the substrate nucleic acid being used to determine enzyme or modifier activity. Alternatively, they may be used in the hereinafter described indirect methods in which the substrate or product molecules of the enzyme or modifier reaction are exposed to a further oligonucleotide sequence prior or subsequent to the reaction.

In the above methodology said substrate or product may be any nucleic acid or sequence thereof but in particular it is gDNA, cDNA, mRNA, tRNA or rRNA.

Moreover, in the above methodology said substrate or product nucleic acid may be single stranded or multi stranded.

In a preferred aspect of the invention an oligonucleotide sequence is labelled with a chemiluminescent molecule that can be rendered non-chemiluminescent by energy transfer quenching, by an energy acceptor, depending on the structure or conformation of the chemiluminescent labelled oligonucleotide sequence. It is well-established that energy transfer occurs when the distance between the emitter and quencher is approximately 5 nanometers or less. Molecules capable of acting as energy transfer donors and acceptors have been described in the literature as has the way in which they can be linked to oligonucleotide probes. Surprisingly we have found that it is possible to use chemiluminescent quenching systems to determine the activity of enzymes or other substances responsible for the metabolism of nucleic acids. This is unexpected since the established chemical and physical conditions required to separately (i) permit the reaction (ii) allow or retain hybridisation and (iii) initiate chemiluminescence are reportedly quite different and it is not clear how to facilitate any one or more of these processes in combination without having a deleterious effect on the other processes. Moreover though chemiluminescent molecules have been used as labels for oligonucleotide probes to detect the presence of target molecules, there is no indication that they can be used to discriminate between the substrate and products of enzymes or other substances responsible for nucleic acid metabolism and therefore can be used as a basis for means of determining the activity or inhibition of activity of the enzymes or other substances.

Of particular use in indirect determinations of enzyme activity are those labelled oligonucleotide sequences whose conformation changes upon hybridisation. The development of such sequences has been described for fluorescence quenching (WO 97/39008) and more recently for chemiluminescence quenching (WO 01/42497 A2) and it is established that such sequences have been applied to the detection of target nucleic acids. Though such work does not encompass the teachings set forth herein, from these principles one skilled in the art can appreciate how to construct the materials which have utility with the present invention. We have now determined that it is possible also to use such intra-molecular labelled chemiluminescent emitter/quencher oligonucleotide sequences (HICS probes) to discriminate between the substrate and product of the reactions of enzymes or other substances responsible for nucleic acid metabolism and hence determine the activity or inhibition of activity of said enzymes or other substances.

Surprisingly, we have also found that in many cases the enzyme or other substance is capable of functioning even when the substrate nucleic acid possesses label moieties. Thus in these cases it is possible to configure means by which the enzyme or other substance of interest is caused to act upon the labelled substrate to yield a labelled product in such a way that the labelled substrate and labelled product possess different optical properties. This we have referred to herein as a direct assay.

Thus, in one embodiment of the invention said methodology includes the aforementioned labelled oligonucleotide comprising a chemiluminescent molecule and its corresponding quencher molecule. Alternatively, in another embodiment of the invention there is provided an oligonucleotide complementary, at least in part, to said substrate nucleic acid, or its corresponding enzyme or reaction product, wherein said oligonucleotide is labelled with either a chemiluminescent molecule or the corresponding quencher molecule; and the other of said chemiluminescent molecule or its corresponding quencher molecule is provided on said substrate or product nucleic acid. Alternatively, yet again, in a further embodiment of the invention said oligonucleotide is omitted from the above methodology and said chemiluminescent molecule and its corresponding quencher molecule is provided on said substrate or product nucleic acid.

In one embodiment when assaying for ligase activity, there is synthesised a double-stranded nucleic acid sequence in which one of the strands possesses a discontinuity (“nick”). The synthesis of such sequence, capable of acting as a substrate for ligase enzymes, is well-known to one skilled in the art. A solution of the substrate is exposed to the enzyme such that if the enzyme is active, the nick will be repaired (“ligated”). The temperature of the reaction mixture is increased such that all double-stranded nucleic acid is dissociated into single-stranded nucleic acid. The presence of any ligated sequence is then demonstrated by reaction with an intra-molecular labelled chemiluminescent emitter/quencher oligonucleotide sequence (HICS probe). Surprisingly we have found that the hybridisation of the aforementioned labelled sequence to the repaired strand results in loss of quenching activity and thus emission of chemiluminescence when measured in a luminometer whereas quenching is maintained in the presence of the nicked strand. It is presumed that the energetically favourable binding of the HICS probe to the ligated sequence results in a change in conformation of the former with associated loss of quenching activity and thus observation of chemiluminescence emission whereas little or no conformational change occurs in the presence of the unligated sequence. Thus it is possible to determine the relative amounts of ligated and unligated forms of the sequence of interest. In this way it is possible to perform an assay for ligase or nuclease enzymes since the substrate and product molecules differ by being ligated or unligated sequences.

Moreover, these results are even more note-worthy because of the remarkable sensitivity of the assay in being able to detect a single discontinuity or nick. It therefore follows that the assay is extremely discriminating.

In an alternative embodiment it may be desired to use a pre-formed, double-stranded substrate nucleic acid in which the chemiluminescent emitter is incorporated into a nucleic acid sequence of one strand and the quencher label is incorporated into a nucleic acid sequence of the complementary strand in such a manner that chemiluminescence emission is quenched due to the close proximity of emitter to quencher within the double stranded nucleic acid. In a preferred example such a method is used for the assessment of DNA ligase activity or inhibition thereof. In said method the contrived substrate is constructed so as to comprise a discontinuity or nick in one of the strands such that the nick is repaired following the action of an active ligase enzyme. Using established knowledge the desired length nucleic acid strands are synthesised and annealed such that the nicked duplex has a melting temperature different to that of the un-nicked (enzyme-repaired) duplex. There is then selected empirically a temperature at which the strands of the nicked DNA are dissociated whereas the strands of the continuous (un-nicked) DNA are substantially non-dissociated. Thus in an assay for ligase activity a solution of the substrate is first incubated with the enzyme under conditions known to be appropriate for enzyme activity. Following exposure to the enzyme the temperature of the reaction mixture is elevated to the desired melting temperature (Tm) described above and chemiluminescence intensity measured in a luminometer. The presence of significant chemiluminescence indicates that the emitter and quencher are separated due to dissociation of the nucleic strands into single strands indicating that repair of the nick has not taken place which reflects lack of enzyme activity. The relative absence of chemiluminescence indicates the presence of quenching and thus the presence of intact duplex as a result of ligase activity. It thus also follows that chemical compounds capable of inhibiting ligase activity of an otherwise active enzyme can be identified using this method.

Similarly, the same principles are applied to the assay of those enzymes or substances which catalyse the insertion (integrase) or transposition (transposase) of discrete nucleotide sequences within a given gene sequence. Here, use is made of an appropriate labelled oligonucleotide sequence which is capable of hybridising with the product sequence but not the substrate sequence. In this way, not only can the activity of integrase or transposase preparations be assessed but it is possible to determine whether chemical compounds added into the reaction mixture are capable of inhibiting the enzyme activity and may thus have utility as pharmacological agents.

Enzymes or substances of the class exemplified by nuclease, ligase, integrase and transposase all have the common feature of catalysing or producing, respectively, the covalent modification of genetic material. There also exist enzymes or substances which cause changes in the non-covalent structure of the genetic material, such enzymes or substances being exemplified by helicase. Activity of these enzymes or substances results in the formation of sections of unwound nucleic acid. Here, use can be made of the fact that the unwound product nucleic acid sequence produced as a result of the enzyme or substance activity is accessible to binding by a complementary labelled oligonucleotide sequence in contrast to the substrate duplex nucleic acid sequence. In this situation, the accessible portion of the nucleic acid can be revealed by an intra-molecular chemiluminescent emitter/quencher labelled oligonucleotide sequence (HICS) probe in a similar manner to the ligase assay described above. Thus, for example, the presence, or absence, of chemiluminescence emission indicates that unwound sequence is present as a result of helicase activity.

In a particularly preferred example of an assay for helicase activity, there is synthesised a double stranded nucleic acid substrate in which the chemiluminescent emitter is incorporated into a nucleic acid sequence of one strand and the quencher label is incorporated into a nucleic acid sequence of the complementary strand in such a manner that chemiluminescence emission is quenched due to the close proximity of emitter to quencher within the double stranded nucleic acid. The presence of helicase activity then causes the duplex nucleic acid to be unwound hence removing the influence of the quencher moiety which results in emission of chemiluminescence. In this case, chemiluminescence intensity is proportional to helicase activity.

As a variation of the situation when labelled oligonucleotide sequence binding is used subsequent to performing the enzymatic or other reaction it may be appropriate to design the labelled oligonucleotide sequence to bind to the substrate rather than the product of the reaction.

The teachings herein can also be applied to those situations where a nucleic acid product is created from small precursors such as individual bases since clearly the product of this reaction is capable of hybridisation with a labelled complementary oligonucleotide sequence whereas the reactants are not. Examples of enzymes that participate in such a reaction are primase, polymerase and reverse transcriptase. Normal enzyme activity gives rise to a nucleic acid capable of hybridisation with a complementary intra-molecular chemiluminescent emitter/quencher labelled oligonucleotide sequence (HICS probe) and the subsequently formed labelled duplex results in emission of chemiluminescence. Inhibition of the enzyme results in no labelled duplex being formed and hence no chemiluminescence. The subsequent measurement of chemiluminescence is therefore a quantitative indicator of the activity or otherwise of the enzyme concerned.

From the teachings herein one skilled in the art would be able to construct assays for a wide range of enzymes or substances of the types considered herein in the knowledge that structural differences in the nucleic acid substrates and products can be used to selectively modulate the optical properties of the chemiluminescent emitter/quencher molecules used as labels.

Further, according to a further aspect of the invention there is provided the use of the methodology described herein for screening an agent for modulating activity in relation to a selected enzyme or other substance and according to yet a further aspect of the invention there is provided said substance identified by said methodology.

Ideally, said modulating activity is pharmacological and, ideally still, it is anti-bacterial, anti-viral, anti-fungal or anti-neoplastic.

Moreover, the methodology of the invention may be used to detect whether a nucleic acid molecule has been altered so that it exists in either a first or second state.

The choice of luminometer and reagents to bring about the chemiluminescent reaction depends on the nature of the chemiluminescent label being used. In general, initiator reagents are used to bring about the chemiluminescent reaction whilst monitoring any emitted light. Alternatively, if the kinetics of the chemiluminescent reaction are sufficiently slow, the chemiluminescence can be initiated prior to placing a reaction vessel into the luminometer.

The use of chemiluminescent labelled and fluorescent labelled oligonucleotide probes for the detection of defined target sequences is well-established, as are general principles of altering the optical properties thereof. Techniques for practising these methods are published in the literature and one skilled in the art has access to the necessary practical details.

However, a compound of the following general formula is suitable for practising the invention:
a compound of general formula (I) 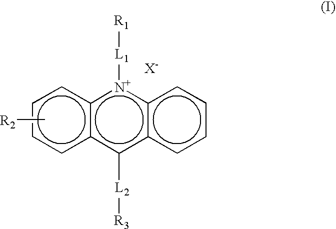
   wherein:
either:
   R₁ is a reactive group capable of reacting with an amine or thiol moiety;

L₁ is a hydrocarbon linker moiety comprising 2-12 carbon atoms, optionally substituted with hydroxy, halo, nitro or C₁-C₄ alkoxy; and

R₂ is hydrogen, C₁-C₄ alkyl, C₁-C₄ haloalkyl, aryl, fused aryl, C₁-C₄ alkoxy, C₁-C₄ acyl, halide, hydroxy or nitro;

or, alternatively:

the combination R₁-L₁— comprises a C₁-C₄ alkyl group optionally substituted with hydroxy, halo, nitro or C₁-C₄ alkoxy; and
R₂ comprises a group R₄-L₁—, where R₄ is a reactive group capable of reacting with an amine or thiol moiety; and L₁ is as defined above;
   L₂ is —C(═O)O—, —C(═O)—S— or —C(═O)N(SO₂R₅)—,
wherein, in each case, the —C(═O) is linked to the ring carbon atom, and R₅ is C₁-C₈ alkyl, aryl, C₁-C₈ alkoxy or C₁-C₈ acyl;

R₃ is a substituted C₁-C₈ alkyl, C₂-C₈ alkenyl, C₂-C₈ alkynyl or aryl group wherein at least one of the said substituents is electron-withdrawing such that the pKa of the conjugate acid of the leaving group formed from R₃ and the —O, —S or —N(SO₂R₅) of the L₂ group is ≦ about 9.5; and

X⁻ is an anion formed as the result of the synthesis and processing of the molecule;

wherein the compound may contain one or more additional R₂ moieties on either or both outer rings, provided that only one of said R₂ moieties may comprise an R₄-L₁-group.

The substituents on the R₃ group are chosen such that the pKa of the conjugate acid of the leaving group formed by R₃ and the —O, —S or —N(SO₂R₅) of the L₂ group is ≦ about 9.5, which in practice means that at least one of the substituents on R₃ is electron withdrawing. This is a particularly important feature as it renders the molecule significantly chemiluminescent at pH 8 or less. Thus, in contrast to some other acridinium compounds, the chemiluminescence emission of the acridinium compounds of general formula (I) can be initiated at pH values compatible with commonly used quenchers and compatible with the stability requirements of ligand-binding complexes.

It will be appreciated that the means of coupling labelling molecules to biologically important molecules are well-established and that there are many variations of R₁ and L₁ which will permit the present invention to be practised. However, it has been found that the favourable results are achieved when L₁ comprises 2 to 10 carbon atoms. More preferably, L₁ is a fully saturated chain consisting of methylene units.

R₁ and R₄ groups which have been found to be particularly useful in linking the compound to a biologically important molecule include active esters, such as succinimidyl esters and imidate esters, maleimides and active halides such as chlorocarbonyl, bromocarbonyl, iodocarbonyl, chlorosulphonyl and fluorodinitrophenyl.

Similarly, it is well-established that there are numerous substitutions that could be represented by R₂ which allow the chemiluminescent activity to be retained and which can be used to alter the emission wavelength of the chemiluminescent label. This affects the choice of quencher used in an assay. The present inventors have found, however, that the compounds which are most useful include those in which R₂ is hydrogen or C₁-C₄ alkyl, especially methyl or ethyl as these are suitable for use in combination with the acceptor methyl red. Compounds in which R₂ is hydrogen are particularly preferred.

In preferred compounds of general formula (I), L₂ is —C(═O)O—.

As already mentioned above, R₃ must be chosen such that the pKa of the conjugate acid of the leaving group formed by R₃ and the —O, —S or —N(SO₂R₅) is ≦ about 9.5 and this means that it contains at least one electron-withdrawing group.

However, if the R₃ moiety forms a leaving group which is too reactive, this will mean that the compound is not always sufficiently stable to be useful as a labelling compound. For optimum efficiency as a labelling compound for chemiluminescent transfer assays, it is therefore preferred that the pKa of the conjugate acid of the leaving group formed by R₃ and the —O, —S or —N(SO₂R₅) of the L₂ group is ≧3.

Therefore, in addition to electron withdrawing groups, the R₃ substituent can contain groups that are not electron withdrawing and may be even electron donating provided that the effect of the electron withdrawing group that is present predominates. The structures of R₃ are readily predictable by calculation of the pKa values of the leaving groups resulting from the chemiluminescent reaction and therefore the scope of such groups can be appreciated by those skilled in the art.

However, by way of example, useful R₃ groups include alkyl or aryl groups substituted with one or more halides or alkyl halides. Particularly preferred compounds include those in which R₃ is a phenyl group substituted independently at the 2 and 6 positions with such groups and particularly with nitro, fluorine, chlorine, bromine or trifluoromethyl. Examples of such groups include 2,6-dibromophenyl, 2,6-bis(trifluoromethyl)phenyl and 2,6-dinitrophenyl.

X⁻ may be any one of a number of suitable anions; however, halide or halide-containing anions such as iodide, fluorosulfate, trifluoromethanesulfonate or trifluoroacetate are preferred.

Compounds where R₂ is hydrogen or lower alkyl are particularly appropriate for use with methyl red as the energy acceptor as these compounds have the particular advantage that their spectrum of chemiluminescence emission lies completely within the absorption spectrum of the quencher methyl red at pH 9. This means that a chemiluminescence energy transfer system in which one of the preferred compounds set out above is used as the emitter and methyl red is the acceptor can be used to make quantitative measurements of the extent and rate of a reaction between a ligand and an anti-ligand and therefore the presence of substances or events that affect the said reaction. This may be done simply by measuring the extent of the change in the chemiluminescent signal.

Particularly preferred compounds for use in the invention include:
9-(2,6-bis(trifluoromethyl)phenoxycarbonyl)-10-(10-succinimidyloxycarbonyl decyl)acridinium trifluoromethanesulfonate;
9-(2,6-dibromophenoxycarbonyl)-10-(10-succinimidyloxycarbonyidecyl)acridinium trifluoromethanesulfonate;
9-(2,6-bis(trifluoromethyl)phenoxycarbonyl)-10-(3-succinimidyloxycarbonyl propyl)acridinium trifluoromethanesulfonate; and
9-(2,6dibromophenoxycarbonyl)-10-(3-succinimidyloxycarbonylpropyl)acridinium trifluoromethanesulfonate;
9-(2,6-dinitrophenoxycarbonyl)-10-(10-succinimidyloxycarbonyldecyl)acridinium trifluoromethanesulfonate; and
9-(2,6-dinitrophenoxycarbonyl)-10-(3-succinimidyloxycarbonylpropyl)acridinium trifluoromethanesulfonate.

The use of luminescent labels also has the advantage that it is possible to configure multichannel assays. There exist, in the literature reports of using both wavelength and temporal discrimination to enable mixtures of labels to be quantified simultaneously, yet independently (U.S. Pat. No. 5,827,656). This same principle can be used to good effect in the present teachings where, for example, it may be desirable to screen chemical compounds simultaneously for inhibitory activity toward, for example, ligase and integrase. Based upon existing knowledge, one skilled in the art would readily appreciate means by which multichannel assays could be demonstrated in the present context.

Additionally, the invention also relates to a nucleic acid for use as a substrate in detecting the activity of a predetermined enzyme or other substance comprising a complex made up of a substrate nucleic acid, a chemiluminescent label and/or a corresponding quencher molecule, said nucleic acid being capable of being acted upon by said enzyme or other substance whereby, on said enzyme or other substance being active, said nucleic acid changes from a first to a second state thereby altering the interaction between said label and its said quencher and so the chemiluminescence emission thereof.

According to a yet further aspect of the invention there is provided an oligonucleotide complementary, at least in part, to a nucleic acid that is to be acted upon by a selected enzyme or substance, or the enzyme or substance product thereof, wherein said oligonucleotide has associated therewith a chemiluminescent label and/or a corresponding quencher molecule; and further wherein said label and said quencher are positioned so that attenuation of chemiluminescence takes place when said oligonucleotide is not hybridised to its complementary sequence.

In a preferred embodiment said oligonucleotide comprises a stem loop arrangement. More specifically, said oligonucleotide comprises a chemiluminescent label located towards a first end of said oligonucleotide chain and a corresponding quencher molecule located towards a second, opposite, end of said chain and further at least one pair of complementary intra-chain sequences which are capable of hybridising theretogether to form a stem loop arrangement.

According to yet a further aspect of the invention there is provided a nucleic acid for use as a substrate in detecting the activity of a predetermined enzyme or substance comprising a chemiluminescent molecule or its corresponding quencher; and an oligonucleotide complementary, at least in part, to said nucleic acid which oligonucleotide comprises the corresponding quencher or chemiluminescent molecule, respectively, to said nucleic acid.

### FIGURE LEGENDS

FIG. 1 is a schematic illustration showing the activity of ligase enzymes;

FIG. 2 is a schematic illustration showing the activity of helicase enzymes;

FIG. 3 is a schematic illustration showing a first DNA ligase assay wherein the substrate is a “nicked” duplex formed by annealing three oligonucleotides, and on the left hand side of the Figure the assay is shown in the presence of a ligase enzyme or a substance with ligase activity and on the right hand side the assay is shown with reference to the absence of ligase enzyme or a substance with ligase activity;

FIG. 4 is an illustration of a further DNA ligase assay using a substrate wherein the duplex is provided with a chemiluminescent molecule and corresponding quencher molecule used to monitor the assay. On the left hand side the assay is shown in the absence of a ligase or an enzyme possessing ligase activity and on the right hand side the assay is shown in the presence of ligase enzyme or an enzyme possessing ligase activity;

FIG. 5 is an illustration of a helicase assay wherein the substrate is a pre-annealed interchain labelled duplex with a ragged end;

FIG. 6 is a schematic illustration of an alternative helicase assay wherein the substrate is pre-annealed duplex, and a further oligonucleotide, labelled with a chemiluminescent molecule and its corresponding quencher, is also used. The oligonucleotide is designed so as to be complementary to one strand of the duplex;

FIG. 7 is a schematic illustration of an RNA polymerase assay wherein the substrate is a DNA duplex containing a promoter operatively linked to a reporter region which encodes a target molecule for a labelled oligonucleotide probe;

FIG. 8 is a schematic illustration of a DNA polymerase assay wherein the substrate is a pre-primed template containing a single stranded coding region for a promoter and a reporter sequence. Further shown in this scheme is the use of an oligonucleotide probe, which is labelled with a chemiluminescent label and a corresponding quencher, and which oligonucleotide is complementary to the transcription product of the reporter region of the nucleic acid molecule;

FIG. 9 is a schematic illustration of a DNA primase assay wherein the substrate is a single stranded DNA containing a DNA primase recognition site upstream of a region encoding a promoter and reporter sequence. In this illustration an oligonucleotide probe, which is labelled with a chemiluminescent molecule and a corresponding quencher molecule, is designed so that it is complementary to the messenger RNA corresponding to the reporter sequence;

FIG. 10 is a graph showing the activity of RNA polymerase over time at an enzyme concentration of 1.1 nM and a substrate concentration of 1 nM;

FIG. 11 is a graph showing the activity of RNA polymerase over time at an enzyme concentration of 0.1 nM and a substrate concentration of 1 nM; and

FIG. 12 is a graph showing the time of a T7 RNA polymerase reaction resulting in the generation of lac z mRNA.

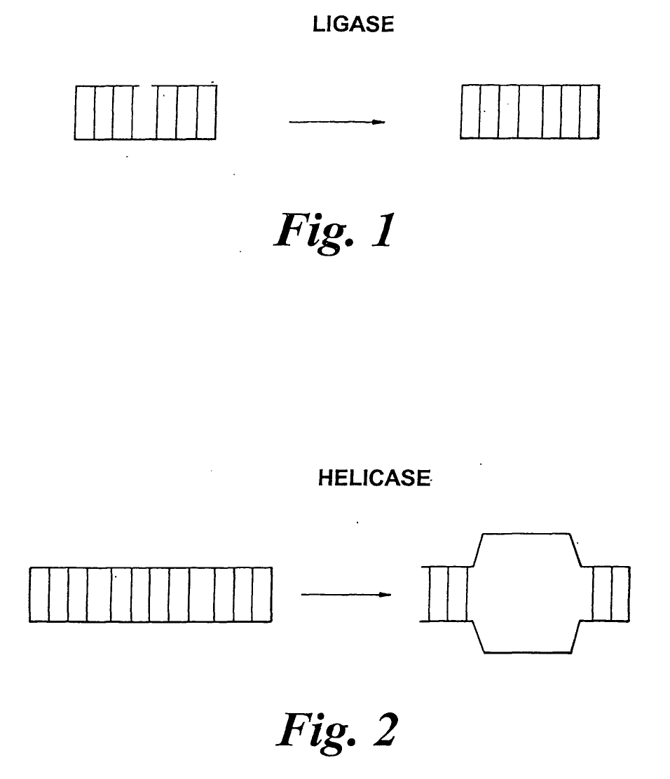

### DETAILED DESCRIPTION OF EMBODIMENTS OF THE INVENTION

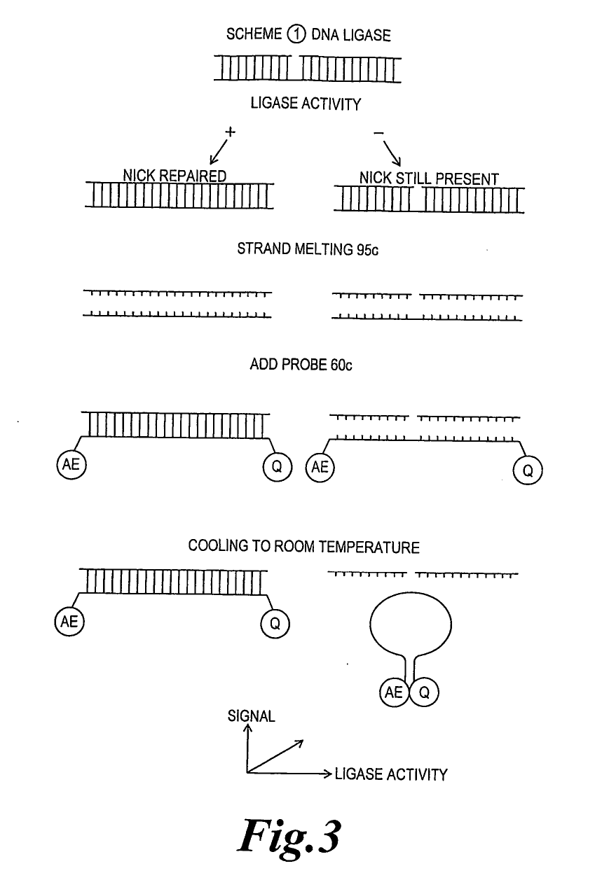

In a preferred aspect of the invention there is produced an assay for the determination of ligase activity. This assay is best illustrated with reference to FIG. 3. Here, a first oligonucleotide sequence is synthesised which comprises a sequence of nucleotides complementary to a second sequence said second sequence, which when bound in a nucleic acid duplex with the first oligonucleotide sequence, can exist either as an intact (ligated) or nicked (unligated) strand. The unligated strand represents at least part of a sequence capable of acting as a ligase enzyme substrate which is converted to the ligated strand by the action of the enzyme and is nicked, preferably, at a position where the ratio of the relative lengths of the two components of the unligated sequence does not exceed four. However, one skilled in the art will appreciate that the possible range of positions of the nick is constrained by the overall length of the nicked sequence. There is also synthesised a third oligonucleotide sequence which comprises at least two linker moieties to which can be attached a chemiluminescent emitter molecule and a quencher molecule, respectively. The positions of the linkers, and label, moieties are arranged such that chemiluminescence quenching occurs when the labelled oligonucleotide is not bound to a complementary nucleic acid and no quenching occurs when the labelled oligonucleotide undergoes a conformational change due to binding to a complementary sequence. The design and synthesis of such labels is well established (WO 01/42497 A2). The sequence of nucleotides of the third oligonucleotide sequence can be complementary to either of the first or second sequences depending on which of the modes of invention described below is practised. In our preferred method, the first and third nucleotide sequences comprise between 10 and 60 bases, more preferably between 20 and 40 bases. Preferably the emitter molecule is a chemiluminescent molecule, more preferably the emitter molecule is a chemiluminescent acridinium salt.

A suitable ligase substrate is prepared by admixture of said first and second sequences such that, following annealing, a nicked duplex is produced. Means of producing such duplexes are well established. In practice the second sequence comprises two shorter sequences one of which is phosphorylated at its free 5′-end by established methods. Preferably, 10-100 nmol of each sequence are hybridised in suitable buffer, which is, for example, lithium succinate 1-100 mM, 0.1-1 ml for 0.5-2 hours at 60° C. A suitable amount of this substrate is then admixed with the desired amount of enzyme and the reaction allowed to proceed for an appropriate period of time under conditions known to be compatible with the particular enzyme being used.

In one mode of the method of the invention (left hand side of FIG. 3), the labelled third oligonucleotide sequence is complementary to the second oligonucleotide sequence. The labelled third oligonucleotide sequence is dissolved in a buffer medium which is compatible with the labelled sequence in terms of allowing it to hybridise to the complementary, intact second oligonucleotide sequence and in terms of maintaining the stability of the reagents during the hybridisation reaction. The formulation of such buffers is established in this field. Typically the buffer ions consist of organic and/or inorganic salts preferably at concentrations in the range 1 to 100 mM and the solutions may contain other solutes such as surfactants and/or preservatives and possess pH values preferably of seven or less. The amount of labelled third oligonucleotide used depends on the sensitivity of detection of the label and the sensitivity of detection of said intact second oligonucleotide sequence required in the assay. The amount of labelled oligonucleotide used for an individual determination is in the range 10⁻¹⁸ to 10⁻⁹ mol, more preferably 10⁻¹⁵ to 10⁻¹² mol. This is contained in a volume of buffer in the range 1 μl to 1 ml, though may be less than 1 μl in certain situations. The solution of labelled oligonucleotide is admixed with the analytical sample in a suitable reaction vessel which is typically a test tube, or part of an array of reaction vessels such as a 96, 384 or 1536 well microtitre plate. Alternatively it is known that many analysis procedures make use of solid-phase systems involving the use of immobilised microarrays and it will be appreciated that the means described herein can be extended to such systems in parallel to the manner in which conventional labelled probe assays have been used.

Following the incubation of the substrate with the enzyme, the temperature of the reaction mixture is increased such that both unligated and ligated duplexes are melted that is, both melting temperatures (Tm) are exceeded. The choice of temperature is determined according to established criteria and methods. The labelled third oligonucleotide sequence is added and the temperature of the reaction mixture reduced to below the Tm of the intact duplex but above the Tm of the nicked duplex.

The incubation with the labelled third oligonucleotide sequence is allowed to proceed for a period of time, preferably, in the range 1 minute to 240 minutes, more preferably 5 minutes to 30 minutes. During this time, any ligated second oligonucleotide sequence formed as a result of ligase enzyme activity will hybridise with the labelled third oligonucleotide sequence and bring about a conformational change in the latter resulting in the presence of chemiluminescence emission when the chemiluminescent reaction is ultimately initiated. Conversely, unligated second oligonucleotide sequence will be incapable of hybridising to the labelled third oligonucleotide sequence to bring about the conformational change of the labelled third oligonucleotide sequence at this reaction temperature which will result in no chemiluminescence being observed when the chemiluminescent reaction is ultimately initiated. Following the hybridisation step, the reaction mixture is allowed to equilibrate to ambient temperature and chemiluminescence activity is measured in a luminometer. In this way, the intensity of chemiluminescence emission is proportional to the ratio of ligated to unligated nucleic acid.

In order to use the above means to determine ligase activity, the hybridisation reaction is preceded by a reaction step in which the enzyme, if present, acts to convert the substrate to the product which is then subjected to the change of temperature and hybridisation conditioris. Where it is desired to determine whether or not a compound or mixture of compounds is capable of inhibiting or activating the enzyme activity, the enzyme is exposed to the compound or mixture of compounds and its activity, or lack thereof, as assayed is compared with the assayed enzyme activity of enzyme not so exposed. In a similar manner the activity of any non-protein molecule or substance causing the conversion of substrate to product can be determined as can the activity of inhibitors or activators thereof.

The method of initiation of the chemiluminescent reaction is dependent on the particular chemiluminescent label being used, such methods being known to those skilled in the art. In the preferred aspect where the label is a chemiluminescent acridinium salt the initiation is typically effected by the addition of hydrogen peroxide and alkali. A wide range of suitable instruments (luminometers) for chemiluminescence detection are commercially available.

The experimental conditions described above are typical of those generally used in the art but other modes of practising the present invention may be used. However, the conditions described herein are an indication of typical practices and are not intended to be restrictive in terms of the wide range of experimental conditions which can be used to practise the invention. One skilled in the art will appreciate that the conditions used must not cause non-specific dissociation of any binding complexes formed prior to initiation of chemiluminescence. Further, one skilled in the art will appreciate that such conditions must not cause a deleterious change in the chemical or optical properties of the chemiluminescent or quencher labels. The design of such conditions is described in the literature.

In another mode of the method of the invention the nucleotide sequence of the third labelled oligonucleotide is complementary to that of the first oligonucleotide sequence. In this mode the reaction mixture resulting from the enzyme incubation is heated to a temperature exceeding the Tm of unligated duplex but below the Tm of ligated duplex and the labelled third oligonucleotide is added. The reaction mixture is incubated at this temperature so as to allow hybridisation to occur to single stranded first oligonucleotide sequence, if present. In this case, the labelled third oligonucleotide sequence is capable of hybridising to the first oligonucleotide sequence such that a conformational change is induced in the former which results in the observation of chemiluminescence when the chemiluminescent reaction is ultimately initiated.

It will be appreciated that in an assay for measuring the activity of an enzyme which facilitates the interconversion of ligated and unligated nucleic acids, the above procedures will be preceded by a method in which the aforementioned enzyme is mixed with the nucleic acid substrate under conditions and in the presence of any co-factors necessary for the reaction to proceed. Also at this point, or prior to this point, there may be added a substance to be investigated as to its possible effect on the activity of the enzyme. The reaction conditions compatible with the activity of a given enzyme are well established in the literature and can be applied to the teachings herein. Moreover the general procedures which represent the best mode for bringing about the interactions between enzymes and inhibitors are well-known. In this context, one skilled in the art will appreciate that the present teachings allow for the study of any agent which will affect the activity of the enzymes or substances described herein. The method of initiation of the chemiluminescent reaction and subsequent measurement of intensity is dependent on the particular chemiluminescent label being used, such methods being known to those skilled in the art. In a preferred method where the label is a chemiluminescent acridinium salt the initiation is typically effected by the addition of hydrogen peroxide and alkali. A wide range of suitable instruments (luminometers) for chemiluminescence detection are commercially available.

Ultimately, the intensity of chemiluminescence is, related to the ratio of the concentration of ligated to unligated sequence and as such is a measure of the activity, inactivity or inhibition of activity of the enzyme present in the system. It will be apparent to the skilled person that the teachings herein can be used as means of determining the activity of a range of enzymes or modifiers which are responsible for the modification of nucleic acid and which involve ligation, and/or cleavage, as part of their overall function. In this situation, one skilled in the art would appreciate the need to optimise the temperature to permit unligated duplex to melt and yet allow ligated duplex to remain intact. Appropriate temperatures will be different for different sequences and an empirical approach is required to optimise this temperature for a given sequence.

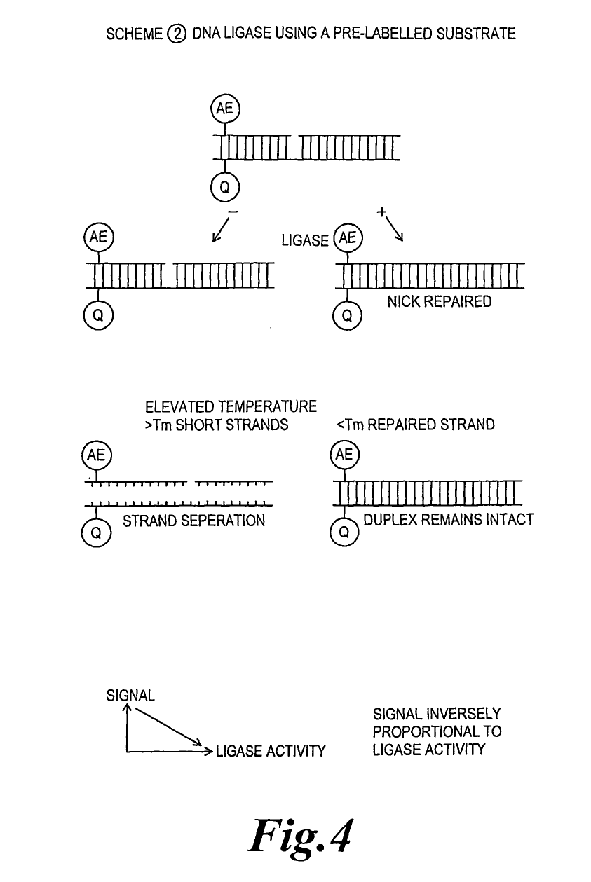

In a further example of a ligase assay (see FIG. 4) using the invention disclosed herein there is produced a contrived ligase substrate consisting of a double-stranded oligonucleotide sequence wherein at least one of the strands possesses at least one nick. The substrate also possesses a chemiluminescent emitter/quencher pair with each label respectively linked via a linker moiety on each strand such that when the oligonucleotide sequence is in double stranded form the chemiluminescence emission is quenched due to the close proximity of the emitter/quencher pair. The design and synthesis of such a contrived sequence is within the knowledge of the skilled man given the established art. Broadly the design and preparation of such contrived substrates follows the guidance outlined above given for the indirect ligase assay except that the first and second oligonucleotide sequences comprising the double stranded substrate carry the labels. In this example, the action of the ligase enzyme, or other ligase like modifier, results in the conversion of unligated substrate to ligated product. When subjected to a temperature sufficient to melt off the unligated strand but below that required to melt off the ligated strand, only the unreacted substrate will melt and in so doing will bring about separation of the emitter/quencher pair resulting in the observation of chemiluminescence when the chemiluminescent reaction is ultimately initiated. It is possible to locate the emitter/quencher pair at mutually opposite locations either at the oligonucleotide terminil or within the duplex itself depending on which position is determined empirically to be optimal for enzyme activity.

Ideally, the amount of contrived labelled substrate used for an individual determination is in the range 10⁻¹⁸ to 10⁻⁹ mol, more preferably 10⁻¹⁵ to 10⁻¹² mol. This is contained in a volume of buffer in the range 1 μl to 1 ml, though may be less than 1 μl in certain situations. The solution of contrived substrate is admixed with the analytical sample in a suitable reaction vessel which is preferably a test tube, or part of an array of reaction vessels such as a 96, 384 or 1536 well microtitre plate. Alternatively it is known that many analysis procedures make use of solid-phase systems involving the use of immobilised microarrays and it will be appreciated that the means described herein can be extended to such systems in parallel to the manner in which conventional labelled probe assays have been used. The enzyme or modifier under study is mixed with the substrate under conditions, and in the presence of any co-factors, necessary for the reaction to proceed. Also at this point, or prior to this point, there may be added an agent to be investigated as to its possible effect on the activity of the enzyme or substance. The reaction conditions compatible with the activity of a given enzyme or substance are well established in the literature and can be applied to the teachings herein. Moreover the general procedures which represent the best mode for bringing about the interactions between enzymes, or substances, and their inhibitors are well-known. In this context, one skilled in the art will appreciate that the present teachings allow for the study of any agent that will affect the activity of the enzymes or substances described herein. Ultimately, the intensity of chemiluminescence is related to the ratio of the concentration of ligated to unligated sequence and as such is a measure of the activity, inactivity or inhibition of activity of the enzyme or substance present in the system.

In a further example of the ligase assay, where a contrived substrate is used which is provided with an emitter/quencher pair, a substrate can be used where the emitter/quencher pair are positioned on the same strand of the duplex substrate and where the complementary strand in the duplex is unligated. The presence of the nick does not allow the unligated second oligonucleotide sequence to significantly change the conformation of the labelled third oligonucleotide sequence which results in no chemiluminescence being observed when the chemiluminescent reaction is ultimately initiated. Conversely the conversion of the unligated substrate to ligated product by an active ligase enzyme, or other substance, results in a conformational change in the contrived labelled substrate which results in the emitter/quencher pair becoming spatially separated and the consequent observation of chemiluminescence when the chemiluminescent reaction is ultimately initiated. In an analogous manner to that taught above, this mode can be used to determine the ability of compounds to inhibit ligase enzymes.

It will be apparent that with the knowledge of the basic teachings herein and with knowledge of the practices employed in related fields of molecular biology and enzymology one skilled in the art would be able to determine other modes of practising the invention

Similar experimental protocols are used for the assay of the activity of helicase enzymes, or substances that act in a similar fashion, or inhibitors thereof, except that in the application of indirect assays to these cases the labelled oligonucleotide sequence is designed such that it is capable of binding to unwound genetic material that constitutes the product of the enzyme, or substance's, activity but incapable of binding to substrate as represented by a nucleic acid duplex. Lack of activity as occurs upon inhibition by a chemical compound, or mixture thereof, results in the absence of accessible target for hybridisation of the labelled oligonucleotide sequence.

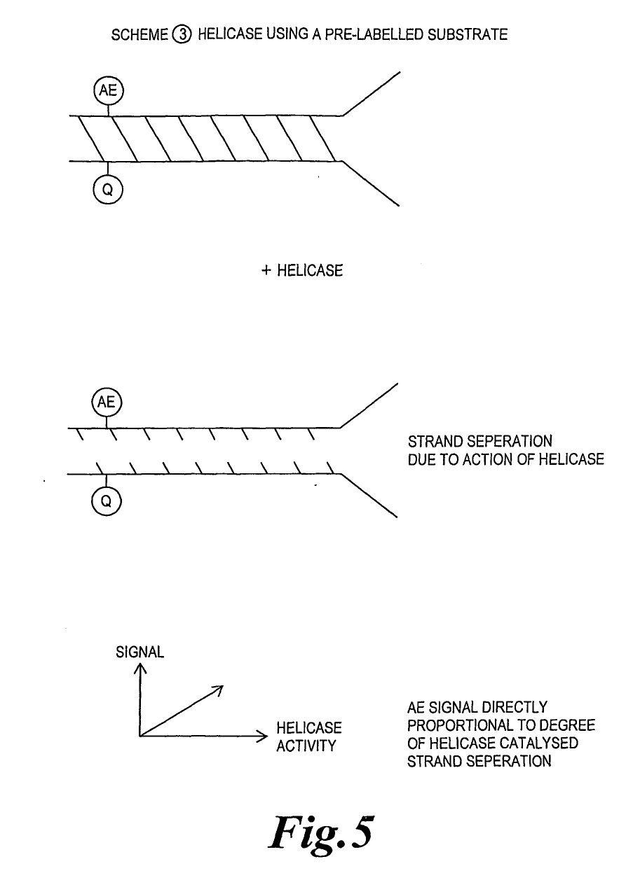

In a further example of a helicase assay (see FIG. 5) a contrived substrate is produced in which each of the strands of the substrate duplex are labelled respectively with partners of the emitter/quencher pair such that the emission intensity of the chemiluminescent label is increased when the duplex has been unwound by an enzyme or substance. In this case the intensity of chemiluminescence is directly proportional to enzyme or substance activity.

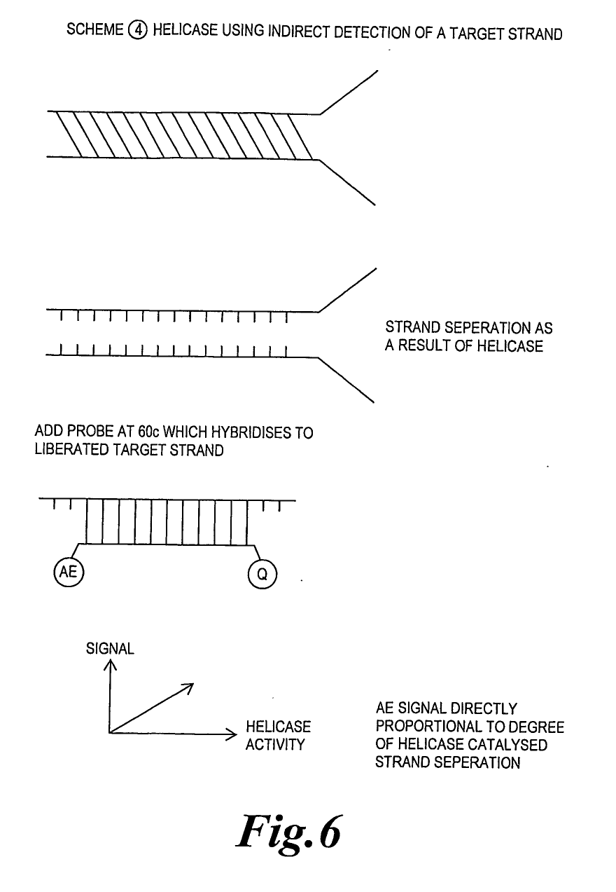

In an alternative example of a helicase assay (see FIG. 6) an oligonucleotide is produced that is complementary to one of the strands of the duplex to be acted upon by the helicase enzyme or a substance of similar activity. The oligonucleotide is further provided with a. pair of linkers in order to couple a chemiluminescent molecule and its corresponding quencher thereto. In this assay, binding of the oligonucleotide to the unwound duplex results in a conformational change that separate the chemiluminescent molecule from its quencher and so results in an increase in chemiluminescence. Thus, in this assay, chemiluminescence is directly proportional to the amount of helicase activity.

In the case of such helicase assays, the design and preparation of substrates and other reagents again are broadly similar to those described above for ligase assays. With this knowledge and the established art, the notional skilled man would be able to design and produce substrates to achieve the desired properties for a given enzyme under study.

Similar experimental protocols are used for the assay of the activity of integrase and transposase enzymes or substance with like activity or, indeed, inhibitors thereof. Here, in indirect assays, labelled oligonucleotide sequences are used that are capable of hybridising to the product nucleic acid sequence but not the substrate nucleic acid sequence or vice versa. Alternatively, in the case of labelled contrived substrates, the intermolecular distance between the emitter/quencher label pair present on adjacent complementary strands is different depending on whether the enzyme or substance is present or absent and, if present, whether it is active or inactive.

Similar experimental protocols are also used for those enzymes or substances which act to increase or decrease the length of nucleotide sequences, for example, polymerase, primase and reverse transcriptase. With the knowledge that the substrates and products of these reactions are structurally distinct, one skilled in the art can apply the teachings herein to carry out assays to determine the activity of these enzymes or substances or, indeed, the inhibitors thereof.

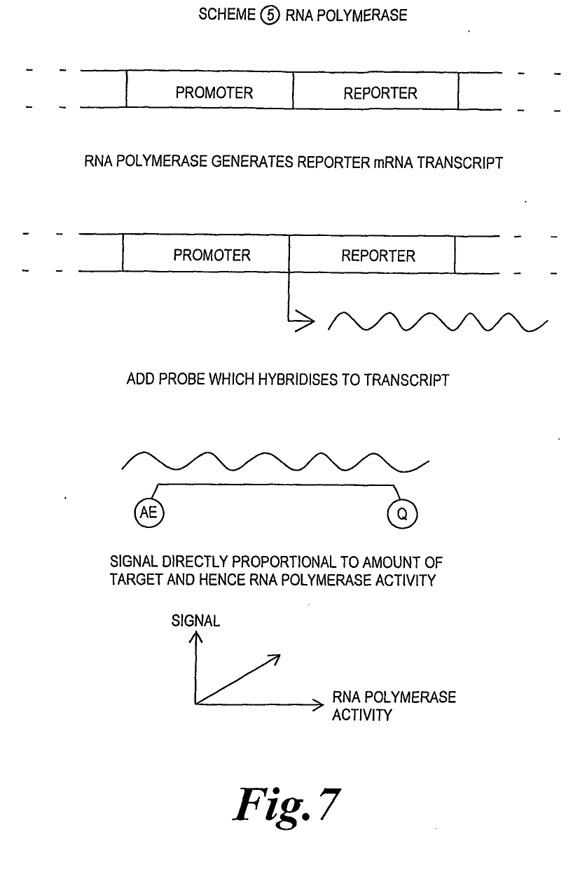

For example, in FIG. 7, there is shown a scheme for assaying an enzyme, or substance, that possesses RNA polymerase activity. The substrate in this reaction is a DNA duplex containing a promoter linked to a region, known as a reporter, coding for a target molecule. In the presence of RNA polymerase transcription occurs and messenger RNA is produced. An oligonucleotide probe which possesses a chemiluminescent molecule and its corresponding quencher molecule is designed to hybridise to said messenger RNA such that when the oligonucleotide probe and the messenger RNA are brought together the probe hybridises thereto. This binding of the oligonucleotide probe to the messenger RNA results in a conformational change that affects the chemiluminescent properties of the probe. The detected signal is directly proportional to the amount of messenger RNA in the sample and thus the activity of RNA polymerase.

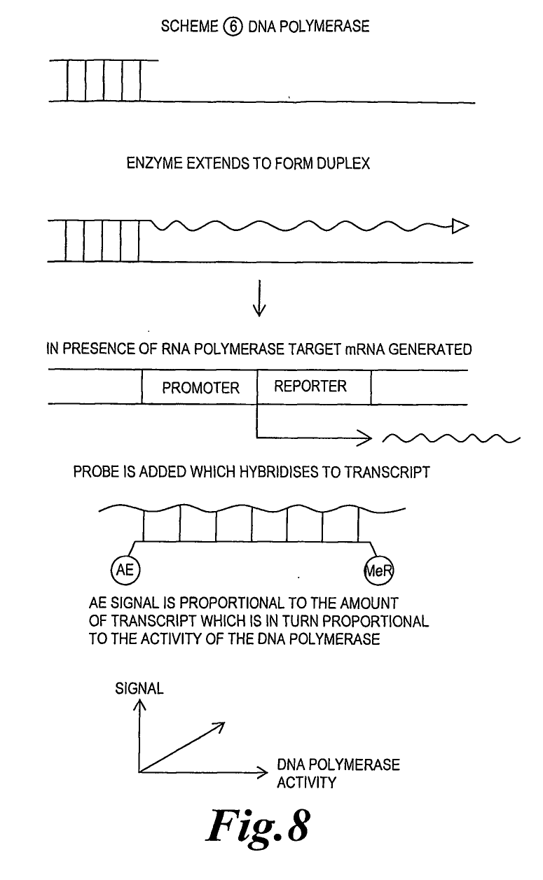

In FIG. 8 there is shown a scheme for assaying for the activity of DNA polymerase. In this example the substrate is a pre-primed template containing a single strand that codes for promoter and reporter sequences. In the presence of DNA polymerase a complementary strand is manufactured to said single strand and thus a duplex is produced. RNA polymerase is then added to the reaction in order to initiate transcription of the reporter region and so bring about the production of messenger RNA. As described with reference to FIG. 7 above, a labelled oligonucleotide is then added to the reaction. This oligonucleotide is designed to hybridise to the messenger RNA and, as mentioned above, the amount of signal is directly proportional to the amount of DNA polymerase that initiated the reaction.

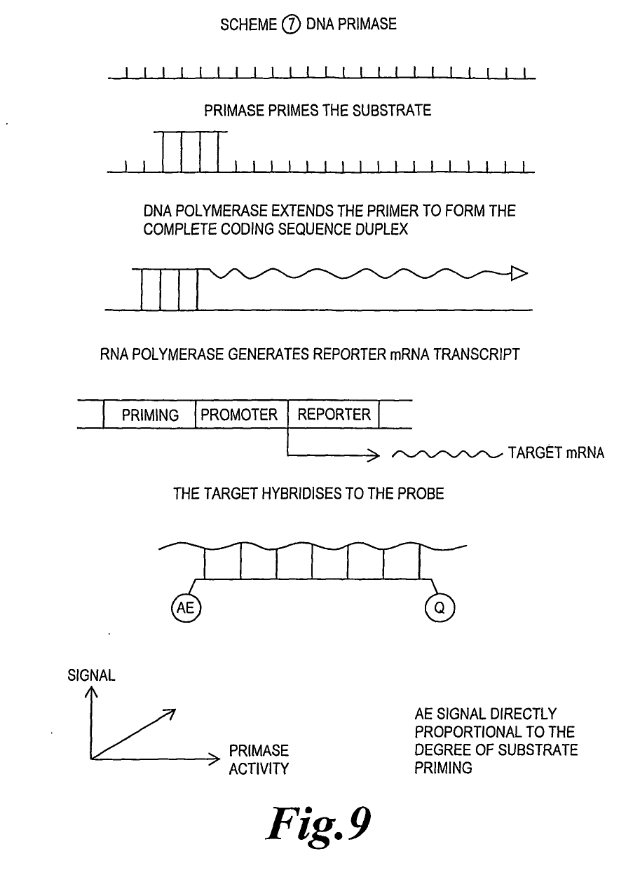

In a further method exemplifying the invention, there is shown in FIG. 9 an assay for DNA primase. In this assay the substrate is a single stranded region of DNA containing a DNA primase recognition site upstream of a promoter and reporter coding sequence. In the presence of DNA primase the primase primes the single stranded substrate. If DNA polymerase is then added to the assay, as mentioned with reference to FIG. 8 above, the DNA polymerase will extend the substrate so as to produce a strand that is complementary to the single strand DNA and so produce a duplex. RNA polymerase is then added to the assay in order to bring about transcription of the duplex and thus the generation of messenger RNA corresponding to the reporter section of the gene. As per the examples illustrated in FIGS. 7 and 8 above, the addition of a labelled oligonucleotide that is complementary to the messenger RNA can then be used to monitor the amount of messenger RNA produced as a result of the reaction. In this example the existence of DNA primase initiates a sequence of events that leads to detection of substrate by the chemiluminescent oligonucleotide probe. Once again, the strength of the chemiluminescent signal is related to the amount of DNA primase present and so able to initiate the reaction.

In each of the examples described above with reference to FIGS. 3 to 9, it will be appreciated that the activity of agents that interfere with the above described reactions can be studied by simply adding a suitable agent to the relevant reaction at an appropriate time and monitoring the effects this has on the chemiluminescent reaction.

Examples of the invention will now be described with particular reference to the following assays.

A hybridisation quench (HyQ) chemiluminescent assay for monitoring DNA ligase activity

This assay utilises a nicked double stranded contrived sequence DNA ligase substrate employing inter-chain labelling with paired chemiluminescent emitter (acridinium ester, AE) as described herein and energy transfer quencher (methyl red, MeR). Removal of the nick by the action of enzyme converts the discontinuous nicked strand to a continuous un-nicked and thus longer strand leading to an increase in its Tm. When subjected to a temperature sufficient to melt off the un-nicked strand but below that required to melt-off the repaired strand, only the unaltered substrate containing the nicked strand will undergo strand separation and in so doing critically deproximate the emitter quencher pair. Following conventional AE chemiluminescence initiation, the resultant signal is directly proportional to the amount of unquenched AE and indirectly proportional to the degree of ligation of the substrate and thus the activity of the DNA ligase. It is possible to locate the AE/MeR emitter/quencher pair at mutually opposite locations either at the chain termini or within the duplex itself.

A synthetic 36 nt oligonucleotide with either a free NH₂ at the 3′ end or linked via an aliphatic side chain at 5 nt from the 3′ terminus was conjugated to 9-(2,6-dibromophenoxycarbonyl)-10-(3-(succinimidyloxycarbonyl)-propyl) acridinium iodide using methods for conventional AE described by Nelson et al. The oligonucleotide was purified by EtOH precipitation and RPLC as described in the above cited reference. Two 18 nt complements were also obtained. The left hand short oligonucleotide (complementary to the first 18 nt of the 5′ end of the AE labelled 36 nt oligonucleotide) was synthesised with a 5′ phosphate to facilitate its reaction with the adjacent base by DNA ligase. The right hand oligonucleotide (complementary to the 18 nt of the 3′ half of the 36 nt oligonucleotide) was obtained commercially conjugated via a linker to methyl red either at its 5′ terminus (for a terminal emitter quencher pair) or via an aliphatic linker 5 nt from the 5′ terminus (for an internal emitter/quencher pair). Annealing of the appropriate combinations (depending on whether an internal or terminal emitter/quencher pair is to be obtained) of the three oligonucleotides by incubating the 36 nt with slight excess of the two respective 18 nt oligonucleotides for 24 to 48 h buffers at room temperature generated the double stranded, nicked ligase substrate. Monitoring of the annealing by measurement of the luminescence demonstrated a time related fall in signal associated with the formation of duplex AE-MeR quenched pair.

For assay of DNA ligase activity suitably diluted substrate how much was incubated at room temperature in the presence of E coli DNA ligase in an assay buffer containing Hepes 200 mM pH 7.2, NaCl 50 mM, MgCl₂ 4 mM, NADH 25 μM (or ATP 10 mM for a non-bacterial DNA ligase), bovine albumin 500 μg/ml, in an assay volume of 10 to 20 μl. At the end of the incubation period the enzyme activity was terminated by the addition of 100 μl of stop buffer (0.05M lithium succinate pH 5.2 containing 8.5% w/v lithium lauryl sulphate) and the assay contents exposed to an elevated temperature, empirically determined to be sufficiently elevated to generate strand separation of the un-nicked but not the nicked MeR conjugated oligonucleotide, for 10 minutes. Endpoint chemiluminescence was then measured using a luminometer with in situ addition of 200 μl 0.2M tris pH 9.0 containing 0.1% H₂O₂, counting for 5 seconds immediately after addition.

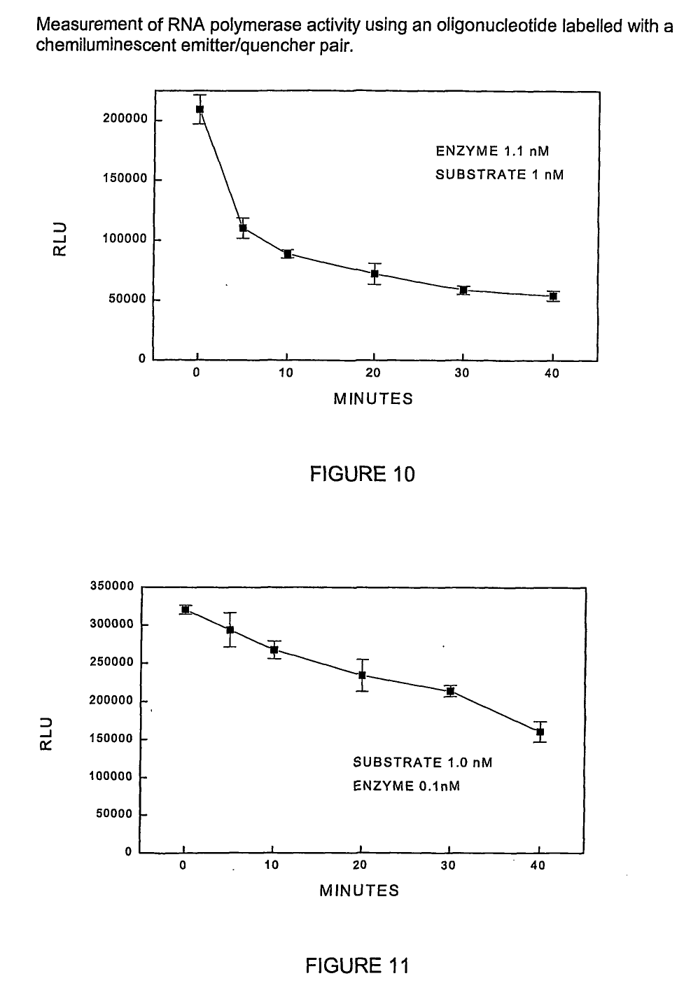

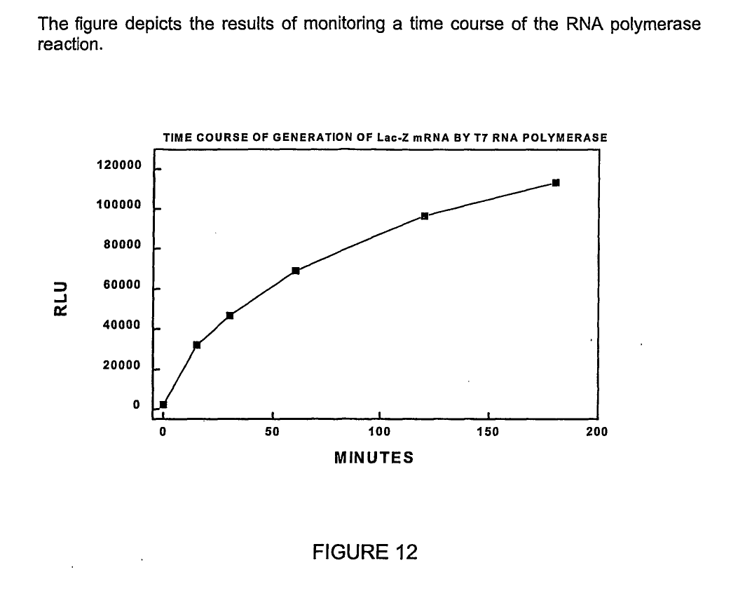

The graphs in FIGS. 10 and 11 depict the time course of substrate (here with a terminal emitter/quencher AE/MeR pair, but equivalent results are obtained with an internally located emitter/quencher AE/MeR pair) turnover in the presence of ligase at two doses of the enzyme. Unrepaired substrate emits luminescence (expressed as RLU) due to separation of the AE/MeR emitter/quencher pair. As substrate is consumed then the proportion remaining in the quenched form following exposure to elevated temperature is increased consistent with the action of ligase repairing the nick and thus preventing strand separation following exposure to elevated temperature.

Principle: this assay utilises a stem loop AE/MeR hybridisation induced chemiluminescence (HICS) probe employing intra-chain terminal labelling with paired chemiluminescent emitter (acridinium ester, AE) as described herein and energy transfer quencher (methyl red, MeR) to monitor the production of specific probe target by the action of T7 DNA dependent RNA polymerase. The probe consists of a synthetic oligonucleotide with the target sequence plus mutually complementary extensions at the 3′ and 5′ termini. The 3′ terminus is covalently linked to acridinium ester (LiAE) and the 5′ linked to methyl red (MeR). When no target is present the single stranded HICS probe is predicted to exist as a stem loop structure such that the AE and MeR are present within the critical energy transfer radius. Upon initiation of the AE chemiluminescence by alkaline peroxide in conditions which maintain probe secondary structure then the chemiluminescent energy is almost entirely absorbed by the MeR quencher resulting in a signal close to background. When hybridised to specific target the probe will undergo linearization and in so doing critically deproximate the emitter quencher pair. Following chemiluminescence initiation, the resultant signal is directly proportional to the amount of unquenched AE and thus directly proportional to the degree of hybridisation, itself proportional to the amount of target. In this example, the target consisted of a 24 nt mRNA sequence of the Lac-Z gene, linked downstream to template T7 promoter and this technique was employed to monitor the generation of target by the action of T7 RNA polymerase.

T7 DNA Dependent RNA Polymerase Generation of lac z mRNA

A linearised section of the commercially available plasmid pGEM-4Z (Promega) was used as source of template for the T7 polymerase. This plasmid contains the T7 polymerase promoter linked to Lac-Z. The desired segment was isolated and amplified using PCR to produce a predicted 336 bp linearised template, which coded for a 295 nt mRNA transcript. Agarose gel analysis of post PCR reaction mixture indicated a single band eluting between the 300 and 400 bp bands of the calibrating ladder. The DNA was extracted from the PCR reaction using a Quiagen kit.

Lac-z RNA transcript was generated using T7 polymerase (0.5 U/μl) plus template (2.5 ng/μl) in an assay buffer containing rNTPs (5 mM) spermidine (2 mM) MgCl₂ (24 mM) RNase inhibitor (0.25 U/μl) and Hepes (80 mM, pH 7.2 with KOH) in an assay volume of 10 μl at an incubation temperature of 37 C.

The enzyme was then stopped using 90 μl of a buffer composed of 85 mM succinic acid, 1.5 mM EDTA, 1.5 mM EGTA, 8.5% lithium lauryl sulphate (pH 5.2 with LIOH) containing Lac-Z HICS probe. The probe consisted of a core 24 nt target sequence plus mutually complementary 3 nt 3′ and 5′ extensions with respectively MeR and AE at the termini. The probe was allowed to hybridise with target for a further 60 minutes at 37 C. Endpoint chemiluminescence was then measured using a luminometer with in situ addition of 200 μl 0.2M tris pH 9.0 containing 0.1% H₂O₂, counting for 5 seconds immediately after addition.

1. A method for determining the activity of an enzyme capable of altering the structure of a nucleic acid from a first state to a second state comprising:
(a) providing in a test sample:
(i) said enzyme;
(ii) said nucleic acid; and, optionally,
(iii) one or more oligonucleotides complementary, at least in part, to said nucleic acid when in said first or second state;
wherein said nucleic acid and/or said oligonucleotide is labelled with at least one chemiluminescent molecule and/or at least one quencher molecule capable of attenuating chemiluminescence from said chemiluminescent molecule, said chemiluminescent and quencher molecules being arranged so that the interaction therebetween changes according to whether said nucleic acid is in said first or second state whereby in one of said first or second states said chemiluminescence is substantially attenuated;
(b) monitoring the chemiluminescence emission of said chemiluminescent molecule; and, optionally,
(c) comparing said emission with that corresponding to the absence of said enzyme.

2. A method according to claim 1 wherein said enzyme is selected from the group consisting of: ligase, nuclease, integrase, transposase, helicase, polymerase, topoisomerase, primase, reverse transcriptase and gyrase.

3. A method according to claim 1 or 2 wherein said nucleic acid is single stranded.

4. A method according to claim 1 or 2 wherein said nucleic acid is double stranded.

5. A method according to claim 1 wherein said chemiluminescent molecule and said quencher molecule are provided on said nucleic acid.

6. A method according to claim 4 wherein said chemiluminescent molecule and said quencher molecule are provided on different strands of said double stranded nucleic acid.

7. A method according to claim 1 wherein said chemiluminescent molecule or said quencher molecule is provided on said nucleic acid, and said corresponding quencher molecule or chemiluminescent molecule is provided on said oligonucleotide.

8. A method according to claim 1 wherein said chemiluminescent molecule and said quencher molecule are provided on said oligonucleotide.

9. A method according to claim 1 wherein said nucleic acid is gDNA, cDNA, mRNA, tRNA or rRNA.

10. A method according to claim 1 wherein included in said test sample is an agent whose ability to affect the activity of said enzyme is to be tested.

11. A method according to claim_10 wherein said agent is added to said test sample either before or after said enzyme.

12. A method according to claim 1 wherein in part (a) thereof a plurality of enzymes and/or a plurality of nucleic acids are provided and, optionally, a plurality of oligonucleotides are also provided wherein said nucleic acids and/or said oligonucleotides are labelled with different chemiluminescent molecules and their corresponding quencher molecules whereby a plurality of chemiluminescent reactions can be simultaneously monitored in order to determine the activity of said enzyme, or a plurality of enzymes, with its, or their, corresponding nucleic acid(s).

13. (canceled)

14. (canceled)

15. A substrate nucleic acid for use in determining the activity of a predetermined enzyme comprising a complex of a nucleic acid, a chemiluminescent molecule and/or a corresponding quencher molecule, wherein said nucleic acid is capable of being acted upon by said enzyme, whereby said substrate nucleic acid changes from a first to a second state thereby altering interaction between said chemiluminescent molecule and said quencher molecule and so the chemiluminescence emission thereof.

16. An oligonucleotide complementary, at least in part, to a nucleic acid that is to be acted upon by a selected enzyme, or complementary to the product of said enzyme's activity, wherein said oligonucleotide has associated therewith a chemiluminescent molecule and a corresponding quencher molecule, and further wherein said chemiluminescent molecule and said quencher molecule are positioned so that attenuation of chemiluminescence takes place when said oligonucleotide is not hybridised to its complementary sequence.

17. An oligonucleotide according to claim 16 wherein said oligonucleotide comprises a stem loop arrangement.

18. An oligonucleotide according to claim 17 wherein said oligonucleotide comprises said chemiluminescent molecule located towards a first end and said corresponding quencher molecule located towards a second end, and further wherein said oligonucleotide comprises at least one pair of complementary intra-chain sequences which are capable of hybridising to form said stem loop arrangement.

19. A chemiluminescent labelling system comprising: a nucleic acid complex for use as a substrate in determining the activity of a predetermined enzyme including a chemiluminescent molecule or its corresponding quencher molecule; and, an oligonucleotide complementary, at least in part, to said nucleic acid, said oligonucleotide comprising the corresponding quencher molecule or chemiluminescent molecule to said nucleic acid.

20. A method for determining the activity of at least one enzyme capable of altering the structure of a nucleic acid from a first state to a second state comprising the steps of:
(a) providing in a test sample:
(i) at least one enzyme whose activity is to be determined;
(ii) nucleic acid(s) for said substance(s); and, optionally,
(iii) one or more oligonucleotides complementary, at least in part, to said nucleic acid(s) when in said first or second state;
wherein said nucleic acid(s) and/or said oligonucleotide(s) is/are labelled with a plurality of chemiluminescent molecules and/or their corresponding quencher molecules, each pair providing an output signal whereby said chemiluminescent and quencher molecules are arranged, with respect to each pair, so that the interaction therebetween changes according to whether said nucleic acid is in said first or second state, whereby in one of said first or second states said chemiluminescence is substantially attenuated;
(b) monitoring the chemiluminescence emission from each of said chemiluminescent pairs; and, optionally,
(c) comparing said emission with that corresponding to the absence of said enzyme.

21. A method for determining the activity of an enzyme that alters the structure of a nucleic acid from a first state to a second state comprising:
(a) providing in a test sample:
(i) said enzyme selected from the group consisting of: ligase, nuclease, integrase, transposase, helicase, polymerase, topoisomerase, primase, reverse transcriptase and gyrase;
(ii) said nucleic acid in a first state; and, optionally,
(iii) one or more oligonucleotides complementary, at least in part, to said nucleic acid when in said first or second state;
wherein said nucleic acid in said first state and/or said oligonucleotide is labelled with at least one chemiluminescent molecule and/or at least one quencher molecule that attenuates chemiluminescence from said chemiluminescent molecule, said chemiluminescent molecule and said quencher molecule being arranged so that interaction therebetween changes according to whether said nucleic acid is in said first or second state, whereby said chemiluminescence is substantially attenuated when said nucleic acid is in one of said first or second states;
(b) detecting the chemiluminescence emission of said chemiluminescent molecule; and, optionally,
(c) comparing said emission with emission generated from a sample in the absence of said enzyme.

22. A method for screening an agent that modulates an enzyme that alters the structure of a nucleic acid from a first state to a second state comprising:
(a) providing in a test sample:
(i) an agent to be tested;
(ii) said enzyme selected from the group consisting of: ligase, nuclease, integrase, transposase, helicase, polymerase, topoisomerase, primase, reverse transcriptase and gyrase;
(iii) said nucleic acid in a first state; and, optionally,
(iv) one or more oligonucleotides complementary, at least in part, to said nucleic acid when in said first or second state;
wherein said nucleic acid in said first state and/or said oligonucleotide is labelled with at least one chemiluminescent molecule and/or at least one quencher molecule that attenuates chemiluminescence from said chemiluminescent molecule, said chemiluminescent molecule and said quencher molecule being arranged so that interaction therebetween changes according to whether said nucleic acid is in said first or second state, whereby said chemiluminescence is substantially attenuated when said nucleic acid is in one of said first or second states;
(b) detecting the chemiluminescence emission of said chemiluminescent molecule; and, optionally,
(c) comparing said emission with emission generated from a sample in the absence of said enzyme or said agent.

23. A method for determining the state of a nucleic acid whose structure can be altered from a first state to a second state by an enzyme selected from the group consisting of ligase, nuclease, integrase, transposase, helicase, polymerase, topoisomerase, primase, reverse transcriptase and gyrase, comprising:
(a) providing in a test sample:
(i) said nucleic acid whose state is to be determined; and
(ii) one or more oligonucleotides complementary, at least in part, to said nucleic acid when in said first or second state;
wherein said nucleic acid and/or said oligonucleotide is labelled with at least one chemiluminescent molecule and/or at least one quencher molecule that attenuates chemiluminescence from said chemiluminescent molecule, said chemiluminescent molecule and said quencher molecule being arranged so that interaction therebetween changes according to whether the nucleic acid is in said first or second state, whereby said chemiluminescence is substantially attenuated when said nucleic acid is in one of said first or second states;
(b) detecting the chemiluminescence emission of said chemiluminescent molecule; and, optionally,
(c) comparing said emission with emission generated from a sample of said nucleic acid in a known first or second state.

24. A substrate nucleic acid for use in the method of claim 21 or 22 comprising a nucleic acid in a first state labelled with a chemiluminescent molecule and/or a quencher molecule that attenuates chemiluminescence from said chemiluminescent molecule, whereby said substrate nucleic acid changes from a first state to a second state when said enzyme acts upon said substrate nucleic acid, thereby altering interaction between said chemiluminescent molecule and said quencher molecule to produce a change in chemiluminescence emission.

25. An oligonucleotide for use in the method of claim 21, 22 or 23, wherein said oligonucleotide comprises a chemiluminescent molecule and a quencher molecule that attenuates chemiluminescence of said chemiluminescent molecule, and further wherein said chemiluminescent molecule and said quencher molecule are positioned so that attenuation of chemiluminescence takes place when said oligonucleotide is not hybridised to a complementary sequence of said nucleic acid.
